(19) United States
(12) Patent Application Publication
(10) Pub. No.: US 2025/0163480 A1
(43) Pub. Date: May 22, 2025

(54) PROCESS FOR BIOTECHNOLOGICAL PRODUCTION OF A BIOPRODUCT

(71) Applicant: COLIPI GMBH, Hamburg (DE)

(72) Inventors: Philipp Arbter, Hamburg (DE); Tyll Utesch, Hamburg (DE); An-Ping Zeng, Rosengarten (DE)

(21) Appl. No.: 18/835,140

(22) PCT Filed: Feb. 1, 2022
(86) PCT No.: PCT/EP2022/052304

§ 371 Date: Aug. 1, 2024

(51) Int. Cl. C12P 7/6436; C12M 1/00; C12M 1/107; C12P 23/00

(52) U.S. Cl. CPC C12P 7/6436; C12M 23/36; C12M 29/06; C12M 47/02; C12M 47/06; C12P 23/00

(57) ABSTRACT

The invention relates to a process and an apparatus for the biotechnological production of a bioproduct. The process involves a first and second bioprocess carried out in a first and second bioreactor (2, 3), and a vessel (4). The first bioprocess is a carbon dioxide producing bioprocess resulting in the formation of the bioproduct, the second bioprocess a chemoautotrophic bioprocess in which CO₂ produced in the first bioprocess is consumed by hydrogen oxidizing bacteria oxidizing H₂ with O₂ as electron acceptor. Electrolytically generated H₂ and O₂ are separated, and the O₂ is fed directly into the second and/or first bioprocess, while H₂ is first dissolved in a medium contained in the vessel (4). H₂ saturated medium is then fed into the second bioprocess. Biomass produced in the second bioprocess is fed into the first bioprocess as an additional C and N source.

The present invention relates to the field of biotechnology, in particular to a process for the biotechnological production of a bioproduct.

Biotechnological production of bioproducts, for example biobased chemicals, is long known and promises the provision of chemicals for commercial use, e.g. in the chemical industry, in a more sustainable and environmentally friendly way. As an example, so-called biofuels like bioethanol or biodiesel have been produced with bioprocesses in order to replace fossil fuels derived from oil (see, e.g., WO 2011/088364 A9, WO 2009/133351 A2). However, considering global warming due to increasing amounts of carbon dioxide (CO₂) in the atmosphere, also bioprocesses for producing chemicals in a large scale will have to minimize release of CO₂ in order to be sustainable.

Both WO 2011/088364 A9 and WO 2009/133351 A2 describe the fixation of CO₂ produced in a first bioprocess in a second bioprocess in order to re-use CO₂ produced. WO 2011/088364 A9discloses a method for converting a carbon source into a lipid, wherein the lipid production is carried out in an aerobic fermenter using the oleaginous yeast Yarrowia lipolytica, and carbon dioxide generated during lipid production is converted into a carbon substrate by CO₂ fixation carried out by bacteria of the genus Clostridium sp. in an anaerobic fermenter. The clostridia use H₂ gas produced by electrolysis of water and fed into anaerobic fermenter as reductant. WO 2009/133351 A2 discloses the production of biofuel using photosynthetic plant cells that fix CO₂ produced, for example, by yeast cells producing biofuel.

WO 2011/139804 A2 describes, inter alia, a method for the capture and fixation of carbon dioxide, e.g. from flue gas, by oxyhydrogen (“Knallgas”) bacteria, wherein oxygen and hydrogen can, for example, be provided by electrolysis of water. Biotechnological production of biofuels using “Knallgas” bacteria is also described in Brigham, C. (2019), Perspectives for the biotechnological production of biofuels from CO₂ and H₂ using Ralstonia eutropha and other ‘Knallgas’ bacteria, Applied Microbiology and Biotechnology 103, 10.1007/s00253-019-09636-y.

It is an object of the invention to improve the biotechnological production of bioproducts, in particular biobased chemicals, in terms of sustainability, in particular in terms of minimizing the net production of carbon dioxide.

In a first aspect the invention provides a process for the biotechnological production of a bioproduct, the process comprising:
a) a first bioprocess comprising cultivation of first microorganisms in a first bioreactor in a first medium at a first pressure p1, the first bioprocess resulting in the formation of the bioproduct and carbon dioxide CO₂,
b) a second bioprocess comprising cultivation of second microorganisms in a second bioreactor in a second medium at a second pressure p2, the second bioprocess being a process producing biomass and consuming molecular oxygen, O₂, molecular hydrogen, H₂, and at least part of the CO₂ produced in first bioprocess of step a),
c) electrolyzing water into O₂ and H₂, feeding at least part of the O₂ produced by electrolysis into the second bioprocess and/or into the first bioprocess, and feeding at least part of the H₂ produced by electrolysis into a vessel containing a head space and a third medium at a third pressure, p3, the third medium being identical to the second medium in the second bioreactor, and
d) feeding at least part of the H₂-containing third medium from the vessel to the second bioprocess,
wherein at least part of the biomass produced in the second bioprocess of step b) is used as a C-source for the first bioprocess of step a), and wherein p3≥p2≥p1.

The process combines two bioprocesses, the first bioprocess being a CO₂ producing process and the second bioprocess being a CO₂ consuming bioprocess, wherein the second bioprocess involves the non-photosynthetic biological fixation of carbon dioxide produced in the first bioprocess by microorganisms oxidizing hydrogen and reducing oxygen, e.g. by “Knallgas” bacteria. The first and second bioprocesses are carried out spatially separated from each other in different bioreactors. CO₂ produced in the first bioprocess is fed into the second bioprocess in order to be at least partly, preferably at least predominantly or completely, consumed in the second bioprocess. The process of the invention is preferably a CO₂ neural process, i.e., a process in which carbon dioxide produced is at least essentially completely consumed, such that no CO₂ is released to the environment. Biomass produced in the second bioprocess with at least part of the CO₂ from the first bioprocess as a carbon source is used as an additional carbon source for the first bioprocess. The biomass may also serve as an additional nitrogen source. Further, while oxygen gas produced by electrolysis of water and used as an electron acceptor by the microorganisms of the second bioprocess is fed directly into the second bioprocess or, alternatively or additionally, into the first bioprocess, the hydrogen gas produced by electrolysis and used as an electron donor by the microorganisms of the second bioprocess is not fed directly into the second bioprocess, but indirectly via a medium contained in a separate vessel. An advantage of this separate supply of oxygen and hydrogen to the second bioprocess is, for example, increased safety in terms of explosion protection. In addition, this also serves to account for the poor solubility of H₂ in aqueous media.

The term “bioproduct” refers to a product of a bioprocess, for example, a chemical compound, in particular organic chemical compound, which is a constituent of, produced by or involves chemical reactions in a living cell, in contrast to, for example, chemicals based on fossil feed stocks. Derived from biomass, biobased chemicals can be structurally identical to existing chemicals derived from fossil sources, or can structurally be different from such chemicals. Examples of biobased chemicals include, for example, fatty acids and fatty acid derivatives, e.g. lipids, alcohols, e.g. diols, and acids, biopolymers or monomers for polymerization, peptides etc. The term also encompasses biomass as a product of a bioprocess, for example living, freeze-dried or hydrolyzed microorganisms. As defined above, the term “biobased chemical” relates to a chemical compound, in particular organic chemical compound, which is a constituent of, produced by or involves chemical reactions in a living cell. The term “biochemical” may also be used synonymously for “biobased chemical”. It should be noted that, although the invention may be mainly described in relation to the production of a biobased chemical, and is also mainly intended for this, this should not be construed as excluding the production of biomass, for example, as a bioproduct.

The term “lipid” relates to organic compounds that are soluble in nonpolar organic solvents (like chloroform or ether) and usually insoluble in polar solvents like water, and includes, for example, fats, oils, waxes, phospholipids, and steroids.

The term “bioprocess” used herein relates to any biological process, i.e. a process involving living cells, in particular living microorganisms like bacteria, or functional components of living cells, for example enzymes, cell-free systems or organelles, to obtain a desired product. The product may be a chemical compound, for example organic chemical compound, a mixture of chemical compounds, for example a mixture of organic chemical compounds, or biomass. Examples are the production of lipids from a substrate like glucose, starch, glycerol, or the like.

A “bioreactor” is understood to mean a reactor that comprises a working volume in which a suitable medium, for example an aqueous medium suitable for supporting growth of a culture of microorganisms, e.g. bacterial cells, can be provided and in which biological and/or biochemical processes and reactions can be carried out, or in which conditions can be created under which biological and/or biochemical processes and reactions take place. The processes and reactions can be material transformations, for example the synthesis, modification or degradation of substances by living cells or a cell-free system, or the growth of biomass, i.e. the proliferation of living cells, for example bacterial cells. Depending on the type of process to be carried out and/or the type of microorganisms used to carry out the process, a bioreactor may, for example, be equipped with facilities for aeration/ventilation and/or agitation of the medium.

The term “closed” in relation to the bioreactor or to the vessel means that the photobioreactor or the vessel are closed to the surrounding environment such that the photobioreactor or the vessel can be pressurized. This does not rule out the possibility that the bioreactor or the vessel has openings, connections or the like, via which, for example, a fluid, e.g. a gas, e.g. CO₂, or a liquid, can be introduced into or removed from the container.

The terms “Knallgas bacteria”, “hydrogen oxidizing bacteria”, “H₂ oxidizing bacteria”, or “oxyhydrogen bacteria”, as used herein, relates to a physiologically defined group of bacteria that is able to grow autotrophically, i.e. to fix carbon dioxide, while oxidizing hydrogen (H₂) and using oxygen (O₂) as terminal electron acceptor. The abbreviation HOB may be used for the terms hydrogen or H₂ oxidizing bacteria. The term “oxyhydrogen” may be used synonymously for “Knallgas”. “Knallgas” is a mixture of gaseous hydrogen and gaseous oxygen. Knallgas bacteria are aerobic facultatively chemolithoautotrophic bacteria. Examples of Knallgas bacteria are Hydrogenophilus thermoluteolus, Hydrogenobacter thermophilus, Hydrogenovibrio marinus, Cupriavidus metallidurans (formerly Alcaligenes eutropha or Ralstonia eutropha), Rhodococcus opacus, Xantobacter autotrophicus, and Cupriavidus necator. The following simplified general reaction scheme describes aerobic CO₂ fixation via H₂ oxidation with O₂ as electron acceptor by Knallgas bacteria: H₂+O₂+CO₂→biomass+H₂O.

The term “chemotrophic” relates to organisms using oxidation of chemical compounds as energy source, as opposed to “phototrophic” organisms, deriving their energy from light. The term “chemolithotrophic” relates to organisms using inorganic chemical compounds, for example hydrogen gas, as electron donor, in contrast to the term “chemoorganotrophic”, which relates to organisms using organic compounds as electron donor. The terms “autotroph” or “heterotroph” relate to the carbon source, CO₂ being the carbon source for “autotrophic” organisms, organic carbon compounds being the carbon source for “heterotrophic” organisms. The term “mixotrophic” may be used for organisms being able to use both organic compounds and CO₂ as a carbon source. The term “chemolithoautotrophic” relates to organisms using inorganic compounds as energy source and electron donor, and CO₂ as a carbon source. The term “facultative chemolithoautotrophic” denotes organisms being able to also grow chemoorganoheterotrophically. “Knallgas” bacteria, for example, grow heterotrophically when H₂ is at tropospheric concentrations and grow chemolithotrophically only when H₂ is available at higher concentrations (Pumphrey G M, Ranchou-Peyruse A, Spain J C., 2011, Cultivation-independent detection of autotrophic hydrogen-oxidizing bacteria by DNA stable-isotope probing, Appl Environ Microbiol. 77(14):4931-4938, doi:10.1128/AEM.00285-11).

The term “yeast” relates to eukaryotic unicellular fungal microorganisms. Examples of yeasts are Saccharomyces cerevisiae, Candida albicans, Yarrowia lipolytica. The term “oleaginous yeast” relates to yeasts that are able to accumulate 20% and more of their cell dry-weight in the form of lipids (e.g. fats; see, for example, Abeln, F., Chuck, C. J., 2021, The history, state of the art and future prospects for oleaginous yeast research, Microb Cell Fact 20, 221doi:10.1186/s12934-021-01712-1; Blomqvist, J., Pickova, J., Tilami, S. K. et al., 2018,Oleaginous yeast as a component in fish feed, Sci Rep 8, 15945, doi:10.1038/s41598-018-34232-x). Examples of oleaginous yeasts are Cutaneotrichosporon oleaginosus, Rhodotorula toruloides, Yarrowia lipolytica, Lipomyces starkeyi, Trichosporon oleaginosus (formerly Cryptococcus curvatus) and Rhodosporidium toruloides.

The term “electrolyzing water” relates to a process in which direct electric current is used to split water into hydrogen and oxygen. The term “electrolyzer” is used to denote an apparatus being able to electrolyze water.

The term “hydrogen” relates, unless otherwise expressly stated or clearly evident from the context, to dihydrogen, i.e. the elemental molecule H2 consisting of two hydrogen atoms joined by a single bond. The terms “molecular hydrogen” or “gaseous hydrogen” may also synonymously be used for H₂.

The term “oxygen” relates, unless otherwise expressly stated or clearly evident from the context, to the elemental molecule O₂ consisting of two oxygen atoms joined by a single bond. The terms “molecular oxygen” or “gaseous oxygen” may also synonymously be used for O₂.

The terms “C-source” or “carbon source” relate to chemical compounds containing at least one carbon atom that can be or is incorporated into chemical compounds constituting cellular material. An example of a C-source, in particular for autotrophic bacteria, is CO₂.

The term, according to which “at least part of the biomass produced in the second bioprocess is used as a C-source for the first bioprocess” is not to be construed as meaning that the biomass is the only carbon source of the first bioprocess. Rather, the biomass serves as an additional carbon source for use in the first bioprocess. Further, the term does also not exclude that the biomass can serve other purposes, e.g. as an additional nitrogen source.

The terms “CO₂ fixation” or “carbon fixation” relate to the biological incorporation of inorganic carbon, in particular in the form of carbon dioxide, into organic carbon compounds, for example carbohydrates. The term “non-photosynthetic biological fixation of carbon dioxide” relates to carbon fixation not involving photosynthesis, i.e. by non-photosynthetic organisms.

The terms “fermentation” or “fermentative” refer to any process in which the activity of microorganisms brings about a desirable change to a chemical compound, foodstuff or beverage.

The expression according to which “the third medium is identical to the second medium” means that the third medium and the second medium have at least essentially the same composition, i.e. essentially the same ingredients in the same concentration. The term does not imply that the concentrations of solved gases, for example the concentrations of H₂, O₂ and CO₂ are identical.

The expression “feeding at least part of the O₂ produced by electrolysis into the second bioprocess and/or into the first bioreactor” includes that all or part of the electrolytically generated O₂ is exclusively fed into the first bioprocess, all or part of the electrolytically generated O₂ is exclusively fed into the first bioprocess, or that at least part of the electrolytically generated O₂ is partly fed into the first bioprocess and partly fed into the second bioprocess, be it simultaneously or intermittently. The terms “part of” or “partly” may refer to a partial flow of a continuous total gas flow, or to portions of an O₂ gas flow divided in a timely manner. A part of a continuous flow of oxygen directed to the second bioprocess may thus be separated from the total oxygen flow and used for oxygen supply of the first bioprocess (see below), and/or the total oxygen gas flow may be fed over a first period of time into the second bioprocess, and over a second period of time into the first bioprocess, for example. The formulation, according to which the oxygen is “directly” fed into the second bioprocess does not exclude that the oxygen is intermediately stored in a storage tank, e.g. a gas cylinder.

An expression according to which “carbon dioxide produced in the first bioprocess is withdrawn from the first bioprocess and fed into the second bioprocess” is not to be construed as meaning that CO₂ is selectively withdrawn from the first bioprocess and fed into the second bioprocess, but as meaning that air containing CO₂ is withdrawn from the first bioprocess, for example from the head space of the first bioreactor, and the CO₂-containing air, optionally enriched or concentrated with CO₂, is fed into the second bioreactor.

In the process of the invention, the electrolytically generated oxygen can, in one embodiment, be exclusively fed into the second bioprocess for the supply of oxygen of the second microorganisms. In an alternative embodiment, the electrolytically generated oxygen can be exclusively fed into the first bioprocess, which, in this embodiment would preferably be an aerobic or at least microaerophilic bioprocess. The oxygen supply of the aerobic second bioprocess would, in this embodiment, be provided via the gas stream containing the CO₂ produced in the first bioprocess, which is withdrawn from the first bioprocess and fed into the second bioprocess. The second bioprocess would thus be supplied with residual oxygen from the first bioprocess. In another embodiment, the electrolytically generated oxygen is partly fed into the first bioprocess and partly fed into the second bioprocess. The oxygen can be divided in terms of quantity or time, i.e. divided into two partial flows fed continuously into the first and second bioprocess or fed intermittently into the bioprocesses.

In a preferred embodiment of the process of the invention, the third pressure p3 is equal to or greater than the second pressure p2, and the second pressure is equal to or greater than the first pressure p1. Particularly preferred, the third pressure p3 is greater than the second pressure p2, and the second pressure is greater than the first pressure p1, i.e. p3>p2>p1. Further preferred, p1 is greater than atmospheric pressure. A greater pressure p3 in the vessel with the third medium into which the H₂ gas is fed leads to a larger amount of H2 that can be solved in the third medium due to the pressure dependence of the solubility of gases. A lower pressure p2 in the second bioreactor compared to the pressure p3 facilitates the release of H₂ into the second medium. The vessel can be configured as a third bioreactor.

The first bioprocess can be any bioprocess resulting in the production of a desired bioproduct and carbon dioxide CO₂. Preferably, the first bioprocess is a bioprocess which does not involve the production of strongly reducing gases, like H₂ or CO. The first bioprocess can be an aerobic, anaerobic or microaerophilic process. Preferably, the first bioprocess is an aerobic or at least microaerophilic process. The bioproduct can be withdrawn from the first bioprocess by any method suitable for the respective bioproduct, e.g. by withdrawing at least part of the cells cultivated in the first bioprocess if the bioproduct is stored within the cells. The cells withdrawn from the first bioprocess may then be further processed to obtain the bioproduct. The first bioprocess may also be a bioprocess for the production of biomass, for example, cell masses of living microorganisms grown in the first bioprocess. In a preferred embodiment of the process according to the invention the first bioprocess is an aerobic bioprocess. Further preferred, the first bioprocess is a fermentation process for the production of lipids. Preferably, the fermentation process involves the cultivation of oleaginous yeast, preferably Rhodosporidium toruloides. Alternatively or additionally, the first bioprocess involves the production of carotenoids, preferably using Rhodosporidium toruloides (see, for example, Igreja, W. S.; Maia, F. d. A.; Lopes, A. S.; Chisté, R. C., 2021, Biotechnological Production of Carotenoids Using Low Cost-Substrates Is Influenced by Cultivation Parameters: A Review, Int. J. Mol. Sci. 22,8819, doi:10.3390/ijms22168819; Yaegashi, J., Kirby, J., Ito, M. et al., 2017, Rhodosporidium toruloides: a new platform organism for conversion of lignocellulose into terpene biofuels and bioproducts, Biotechnol Biofuels 10, 241, doi:10.1186/s13068-017-0927-5). The microorganisms, for example yeast cells, used in the first bioprocess can be genetically engineered.

In case of an aerobic first bioprocess oxygen generated electrolytically can exclusively be fed into the first bioprocess, or can also be partly fed into the first bioprocess in order to support the aerobic process. The latter can be done, for example, in that a) the O₂ gas flow is divided into two gas streams, one of which is fed into the second bioprocess and the other into the first bioprocess, or b) in that the total O₂ gas flow is intermittently fed into the first or the second bioprocess. A combination of both options a) and b) is, of course, also possible. As mentioned above, it is also possible that the oxygen is exclusively fed into the first bioprocess, the second bioprocess being supplied with oxygen via the gas stream containing the CO₂ withdrawn from the first bioprocess.

In a preferred embodiment of the process of the invention, the second bioprocess is a non-photosynthetic bioprocess, and involves the chemolithoautotrophic consumption of O₂, H₂ and CO₂. In this embodiment, the second bioprocess is a bioprocess in which Knallgas bacteria are grown that oxidize H₂ with O₂ as electron acceptor and fix carbon dioxide. Preferably, the carbon dioxide stems at least predominantly, further preferred entirely from the first process. The microorganisms used in the second bioprocess can be genetically engineered.

Particularly preferred the first bioprocess is an aerobic process for the fermentative production of lipids and/or carotenoids involving the cultivation of yeast cells, preferably of the genus Rhodosporidium, particularly preferred Rhodosporidium toruloides, and the second bioprocess involves the chemolithoautotrophic cultivation of Knallgas bacteria, preferably of the genus Hydrogenophilus, particularly preferred Hydrogenophilus thermoluteolus, with H₂ and O₂. O₂, produced by electrolysis, is fed into the second bioreactor, whereas the H₂ from the electrolysis is fed into the vessel where it is dissolved in the third medium, part of which is fed into the second bioreactor in order to supply the hydrogen oxidizing bacteria with H₂. The main carbon source for the first process can, for example, be carbonaceous waste and by-products (e.g. biomass hydrolysates, molasses etc). Other carbon sources, for example glycerol or glucose, are, however, also possible. In this embodiment, the carbon dioxide produced by the yeast cells is fed into the second bioprocess involving the production of biomass of Knallgas bacteria, the Knallgas bacteria growing chemolithoautotrophically. In a preferred embodiment of the process of the invention, the first microorganisms are thus microorganisms of the genus Rhodosporidium, preferably Rhodosporidium toruloides, and the second microorganisms are microorganisms of the genus Hydrogenophilus, preferably Hydrogenophilus thermoluteolus. Other combinations of a Knallgas bacteria for the second bioprocess and yeasts or bacteria for the first bioprocess are, of course, also possible.

In a preferred embodiment of the process according to the invention, the second bioprocess is performed at a temperature between 40-55° C., preferably 45-55° C., 50-55° C. or 50-53° C. This is particularly preferred in case of the cultivation of thermophilic Knallgas bacteria, e.g. Hydrogenophilus thermoluteolus.

In a preferred embodiment of the process of the invention, the third medium in the vessel is kept anaerobic. This can, for example, be achieved in that the medium is not aerated and as a result of the activity of Knallgas bacteria in the medium consuming any oxygen with the H₂ introduced into the medium.

The first, second and third medium are in each case an aqueous medium that is suitable for the intended purpose, for example for supporting growth of the microorganisms cultivated.

Fresh medium for the second bioprocess is preferably fed into the vessel, but may alternatively or additionally also be fed into the second bioreactor.

In a preferred embodiment of the process of the invention, part of the second medium of the second bioreactor containing the second microorganisms is withdrawn from the second bioreactor, the biomass contained in the second medium is at least partly separated from the second medium, hydrolyzed and fed into the first bioprocess, and the second medium separated from the biomass is recycled into the second bioprocess. Alternatively, or additionally, the medium separated from biomass can also be returned to the vessel. In this embodiment the biomass produced in the second bioprocess is used as an additional carbon and nitrogen source for the first bioprocess, i.e., as a substrate for growing the microorganisms cultivated in the first bioreactor. Further, at least part of the biomass produced in the second bioprocess and separated from the second medium can be withdrawn from the whole process and used as a separate bioproduct, e.g. as fish food.

In a further preferred embodiment of the process of the invention, part of the second medium from the second bioreactor is fed into the vessel and sprayed into the head space of the vessel containing the third medium, and an amount of third medium, preferably essentially the same amount as the amount of second medium being fed into the vessel, is fed from the vessel to the second bioreactor. Since the media in the second bioreactor and the vessel are the same, part of the medium in the second bioreactor and the vessel is circulated, preferably continuously, from the second bioreactor to the vessel and back to the second bioreactor. The medium is sprayed into the head space of the vessel, e.g. with the aid of a pump and a nozzle. The pump brings the pressure of the part of the second medium to be fed into the vessel to a pressure >p3 and atomizes the medium in the head space of the vessel. Simultaneously, an amount, preferably the same amount, of third medium in the vessel is returned to the second bioreactor. In this embodiment, part of the second and third medium, including the bacteria therein, is circulated between the second bioprocess and the third bioprocess. The fine distribution of the second medium in the head space of the vessel promotes the transition of H₂ from the gas phase to the liquid phase. In this manner, electrolytically generated H₂ gas fed into the vessel is efficiently dissolved in the medium therein and fed into the second bioprocess.

The process of the invention can be configured as a batch, fed-batch or continuous process. Preferably, the process of the invention is a continuous process, i.e. configured as a continuous process.

Carbon dioxide produced in the first bioprocess is withdrawn from the first bioreactor, e.g. from the head space of the first bioreactor, and fed into the second bioreactor. After withdrawal from the first bioreactor and before its introduction into the second bioreactor, the carbon dioxide is preferably concentrated, for example, by using a suitable separation membrane, for example hollow-fiber membrane, separating N₂ from CO₂ (see, for example, Tong Z, Sekizkardes A K., 2021, Recent Developments in High-Performance Membranes for CO₂ Separation, Membranes (Basel) 11(2):156, doi:10.3390/membranes11020156; Khalilpour R., Mumford K., Zhai H., Abbas A., Stevens G., Rubin E. S., 2015, Membrane-based carbon capture from flue gas: a review, Journal of Cleaner Production 103, 286-300, doi:10.1016/j.jclepro.2014.10.050; U.S. Pat. No. 10,118,136 B2). Suitable membranes are, for example, hollow-fiber membranes “SEPURAN® Green (Evonik Industrie AG, Essen, Germany).

The CO₂ withdrawn from the first bioreactor can optionally be intermediately stored in a storage tank, e.g. a gas cylinder, for example, in case the first bioprocess and the second bioprocess are not running synchronously in terms of carbon dioxide production and consumption, and for a better control of the CO₂ supply to the second bioprocess.

The oxygen generated electrolytically is, in a preferred embodiment, at least partly directly fed into the second bioprocess in order to support the metabolic activity of the Knallgas bacteria. The oxygen may continuously or intermittently be fed into the second bioprocess. The oxygen may intermediately be stored in a storage tank, for example a gas cylinder. The oxygen may also be fed into the second bioprocess depending on physical parameter measured in the second bioprocess, e.g. pH, pO₂, pH₂, temperature, cell mass etc. A flow regulation may be applied in order to control the flow rate of the oxygen fed into the second bioreactor. In this embodiment, the oxygen generated electrolytically can additionally be fed, continuously or intermittently, into the first bioprocess, in particular in case of an aerobic bioprocess. The oxygen flow into the first bioprocess can also be subject to regulation.

In a second aspect the invention relates to an apparatus for the biotechnological production of a bioproduct according to the process of the invention, the apparatus comprising:
a) a first bioreactor being configured for carrying out a first bioprocess, wherein the first bioprocess comprises the cultivation of first microorganisms in a first medium at a first pressure p1, and wherein the first bioprocess results in the formation of the bioproduct and carbon dioxide CO₂,
b) a second bioreactor being configured for carrying out a second bioprocess, wherein the second bioprocess comprises the cultivation of second microorganisms in a second medium at a second pressure p2, and wherein the second bioprocess is a process producing biomass and consuming molecular oxygen O₂, molecular hydrogen H₂, and at least part of the CO₂ produced in the bioprocess of step a),
c) a vessel being configured to contain a head space and a third medium at a third pressure p3, and
d) an electrolyzer for electrolyzing water into O₂ and H₂, and wherein the apparatus further comprises
a first fluid connection fluidically connecting a first outlet of the first bioreactor to a first inlet of the second bioreactor, for the transfer of CO₂ produced in the first bioreactor to the second bioreactor,
a second fluid connection fluidically connecting a first outlet of the second bioreactor to a first inlet of the first bioreactor, for the transfer of biomass produced in the second bioreactor to the first bioreactor,
a third fluid connection fluidically connecting a second outlet of the second bioreactor to a first inlet of the vessel, for the transfer of second medium to the vessel,
a fourth fluid connection fluidically connecting a first outlet of the vessel to a second inlet of the second bioreactor, for the transfer of third medium to the second bioreactor,
a fifth fluid connection fluidically connecting a first outlet of the electrolyzer to a third inlet of the second bioreactor, for the transfer of oxygen O₂ to the second bioreactor,
a sixth fluid connection fluidically connecting a first outlet of the electrolyzer to a third inlet of the first bioreactor, for the transfer of oxygen O₂ to the first bioreactor, and
a seventh fluid connection fluidically connecting a second outlet of the electrolyzer to a second inlet of the vessel, for the transfer of hydrogen H₂ to the vessel.

The apparatus of the invention is configured to carry out the process of the invention and comprises a first bioreactor, a second bioreactor and a vessel, that can also be configured as a bioreactor. The bioreactors and the vessel are fluidically connected to each other in a manner to carry out the process of the invention. The apparatus thus comprises fluid connections, for example pipes or tubes or the like, interconnecting the bioreactors and the vessel fluidically in a manner to allow the transfer of CO₂ produced in the first bioreactor to the second bioreactor, to transfer biomass from the second bioreactor to the first bioreactor, to feed oxygen generated by the electrolyzer into the second bioreactor and/or into the first bioreactor, and hydrogen into the vessel, and to circulate medium between the second bioreactor and the vessel.

In a preferred embodiment of the apparatus of the invention, the second fluid connection comprises, between the first outlet of the second bioreactor and the first inlet of the first bioreactor, a separator for separating the second microorganisms from the second medium, and a hydrolyzer for hydrolyzing the second microorganisms separated from the second medium by the separator. The separator can be a centrifuge, running batchwise or continuously, or a hollow fiber membrane module. Medium separated from the second microorganisms can be returned to the second bioreactor, fed into the vessel or withdrawn from the process.

In a further preferred embodiment of the apparatus of the invention, the third fluid connection fluidically connecting the second outlet of the second bioreactor to the first inlet of the vessel, extends into an upper part of the vessel comprising the head space, the extension comprising a nozzle for atomizing second medium fed into the vessel.

Further preferred, the first fluid connection fluidically connecting the first outlet of the first bioreactor to the first inlet of the second bioreactor, comprises a CO₂ concentrator, preferably a membrane-based CO₂ concentrator, for concentrating the CO₂ produced in and withdrawn from the first bioreactor before it is transferred to the second bioreactor. Still further preferred, the first fluid connection comprises a CO₂ storage tank, for example a gas cylinder, for storing CO₂. CO₂ can thus be intermediately be stored and fed into the second bioprocess when needed. This is advantageous because it allows a decoupling of the CO₂ production in the first bioprocess from the CO₂ consumption in the second bioprocess, for example, in case of process errors or to compensate for unequal CO₂ production and consumption rates.

The sixth fluid connection, fluidically connecting the first outlet of the electrolyzer to the third inlet of the first bioreactor for the transfer of oxygen O₂ to the first bioreactor, shares part of the fifth fluid connection. In a preferred embodiment of the apparatus of the invention, the fifth fluid connection, fluidically connecting the first outlet of the electrolyzer to the third inlet of the second bioreactor, extends to the first bioreactor and fluidically connects the first outlet of the electrolyzer to the third inlet of the first bioreactor. Preferably, a two-way valve is inserted in the fifth fluid connection such that the oxygen flow can be divided and directed to both the first and second bioreactor at the same time, or intermittently directed either to the first or the second bioreactor.

The vessel can also be configured as a bioreactor, such that the apparatus comprises a first, second and third bioreactor.

In the following, the invention will be described in further detail by way of example only with reference to the accompanying figures.

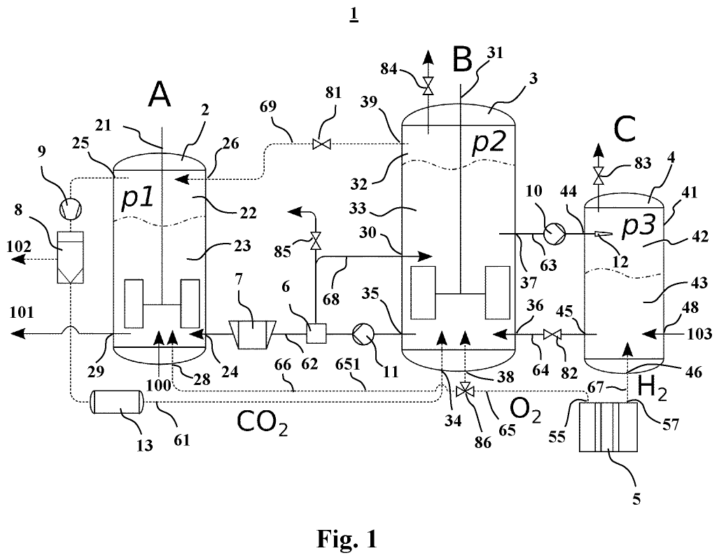

FIG. 1. Schematic illustration depicting an embodiment of an apparatus according to the invention. Gas streams in dashed lines, liquid streams in solid lines.

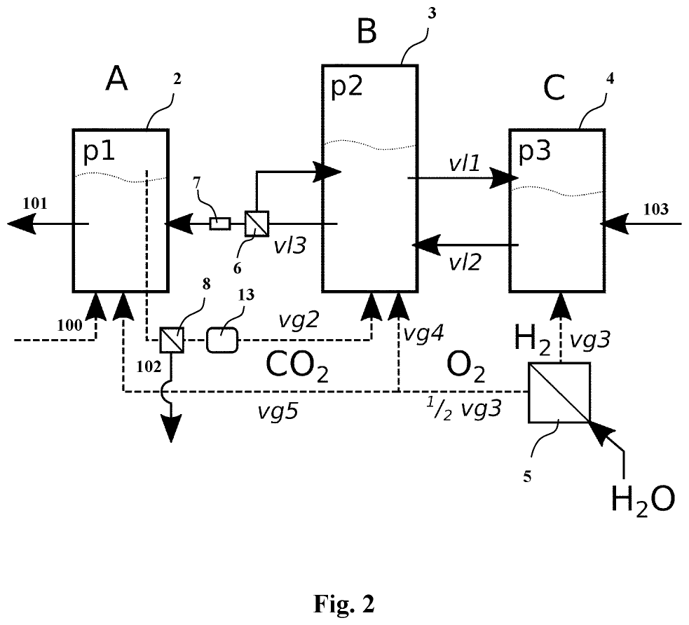

FIG. 2. Simplified illustration of the embodiment of an apparatus according to the invention shown in FIG. 2 with gas and liquid streams. Gas streams in dashed lines, liquid streams in solid lines.

FIG. 1 shows a simplified schematic representation of an apparatus 1 of the invention. The apparatus comprises three sections A, B and C. Section A comprises a first bioreactor 2, section B a second bioreactor 3, and section C a vessel 4, which may be configured as a third bioreactor. The first bioreactor 2 and second bioreactor 3 are equipped with a first stirrer 21 and a second stirrer 31 for agitating the first medium 23 in the first bioreactor 2 or the second medium 33 in the second bioreactor 3, respectively. A first pressure p1 prevails in the first bioreactor 2, a second pressure p2 in the second bioreactor 3 and a third pressure p3 in the vessel 4.

In section A, comprising the first bioreactor 2, a first bioprocess is carried out, the first bioprocess being a bioprocess in which a desired biobased chemical is formed by first microorganisms cultivated in the first bioreactor 2, accompanied by the formation of carbon dioxide, CO₂. An example of such a first bioprocess is an aerated yeast fermentation with Rhodosporidium toruloides for variable lipid production. Here, carbon-containing waste materials and by-products (biomass hydrolysates, molasses, etc.), for example, are used as the primary C source. In addition to lipids, R. toruloides produces carotenoid-containing biomass, which can be used as feed in aquaculture (shrimp, salmon farms; see, e.g., Blomqvist, J., Pickova, J., Tilami, S. K. et al., 2018, Oleaginous yeast as a component in fish feed, Sci Rep 8, 15945, doi:10.1038/s41598-018-34232-x) or as a coloring agent in the food industry (see, e.g., Igreja, W. S.; Maia, F. d. A.; Lopes, A. S.; Chisté, R. C., 2021, Biotechnological Production of Carotenoids Using Low Cost-Substrates Is Influenced by Cultivation Parameters: A Review, Int. J. Mol. Sci. 22, 8819, doi:10.3390/ijms22168819). The lipid profile can be adjusted by genetic modifications of R. toruloides (Wen Z., Zhang S., Odoh C. K., Jin M., Zhao Z. K., 2020, Rhodosporidium toruloides—A potential red yeast chassis for lipids and beyond, FEMS Yeast Research, Volume 20, Issue 5, foaa038, doi:10.1093/femsyr/foaa038). The lipid profile produced can be variably adapted for the cosmetics/food industry so that the lipids can, for example, be classified as palm oil-like to cocoa butter-like.

The first bioreactor 2 containing a first medium 23 with first microorganisms, here, for example, cells of the oleaginous yeast Rhodosporidium toruloides, is aerated with air 100. Alternatively or additionally, the first bioreactor 2 can be supplied with oxygen generated in an electrolyzer 5 electrolyzing water. The exhaust gas containing CO₂ produced in the first bioreactor 2 is withdrawn from the head space 32 of the first bioreactor 2 by means of a compressor 9 and fed via a first fluid connection 61 into the bioprocess carried out in a second bioreactor 3 in section B as a C source. The first fluid connection 61, which may, for example, be a pipe or tube, is fluidically connecting a first outlet 25 of the first bioreactor 2 to a first inlet 34 of the second bioreactor 3. On its way to the second bioreactor 3, the CO₂ is concentrated by means of a CO₂ concentrator 8, for example, a membrane-based CO₂ concentrator 8, so that CO₂-depleted air 102 containing mainly N₂ is removed from the exhaust gas stream. The gassing rate vg2 (see FIG. 2) with which CO₂, or, more precisely, air enriched with CO₂, is fed into the second bioreactor 3 is thus set by the aeration rate, i.e. the rate with which air 100 is fed into the first bioreactor 2, and the compressor 9. In the embodiment shown, the first fluid connection 61 further comprises, here, in the direction of the flow of the CO₂, after the CO₂ concentrator 8, a storage tank 13 for intermediately storing CO₂. A bioproduct 101, for example a biobased chemical, produced in section A, e.g. a chemical compound being excreted into the first medium 23 or accumulated in the cells grown in the first bioprocess, can be withdrawn from the bioprocess in section A.

In section B, second microorganisms, namely chemolithotrophic so-called “Knallgas bacteria”, are grown in a second bioreactor 3 on CO₂, H₂ and O₂. The second microorganisms cultivated in section B can be any microorganisms with the specified gas requirements. An example of a suitable microorganism is Hydrogenophilus thermoluteolus, which has a particularly high growth rate (Arai H, Shomura Y, Higuchi Y, Ishii M., 2018, Complete Genome Sequence of a Moderately Thermophilic Facultative Chemolithoautotrophic Hydrogen-Oxidizing Bacterium, Hydrogenophilus thermoluteolus TH-1, Microbiol Resour Announc.7(6):e00857-18, doi: 10.1128/MRA.00857-18). The bioprocess in section B receives CO₂ and residual O₂ from the associated bioprocess in section A, as well as pure H₂ and O₂ from an electrolyzer 5, as explained in more detail below.

O₂ and H₂ used by the hydrogen oxidizing bacteria grown in the second bioreactor 3 are generated by an electrolyzer 5. O₂ is directly fed into the second bioreactor via a fifth fluid connection 65 fluidly connecting a first outlet 55 of the electrolyzer 5 and a third inlet 38 of the second bioreactor 3. In the embodiment of an apparatus 1 of the invention shown in FIG. 1 an extension 651 of the fluid connection 65 fluidly connects a third inlet 28 of the first bioreactor 2 with the fluid connection 65 and thus with the first outlet 55 of the electrolyzer 5, such that O₂ generated by the electrolyzer 5 can also be fed into the first bioreactor 2 via a sixth fluid connection 66, the sixth fluid connection 66 sharing part of the fifth fluid connection 65. A two-way valve 86 may, for example, be used to divide the O₂ gas stream into a first part, vg4 (see FIG. 2), being fed into the second bioreactor 3, and a second part, vg5, being fed into the first bioreactor 2 via extension 651. The valve 86 may alternatively be used to direct the O₂ stream exclusively into the first bioreactor 2, or to direct the O₂ stream intermittently into the second or the first bioreactor 2. The O₂ gas may be stored intermediary in a separate storage tank (not shown).

Due to the significantly poorer solubility of H₂ in the aqueous medium used, and in order to separate gas phases containing O₂ or H₂ for avoiding flammable spaces and thus create explosion protection, H₂ is not directly fed into the second bioreactor 3. Rather, the H₂ entry is relocated to section C comprising the vessel 4 containing a third medium 43, which, however, corresponds to the second medium 33 in the second bioreactor 3. H₂ gas is fed into the vessel 4 via a seventh fluid connection 67 fluidly connecting a second outlet 57 of the electrolyzer 5 with a second inlet 46 of the vessel 4. The H₂ gas may also be stored intermediary in a separate storage tank (not shown). The electrolyzer 5 builds up an overpressure in the vessel 4. In this embodiment, the pressure p3 prevailing in the vessel 4 is the highest pressure, compared to the pressures p1 and p2 in the other sections, i.e. sections A and B. The increased pressure provides a higher solubility coefficient of H₂. The pressure is adjusted via the electric current I applied to the electrolyzer 5 (proportional to the H₂ gassing rate vg3, see FIG. 2) and the position (degree of closure) of the second valve 82. The medium cycling between sections B and C has an additional positive effect on the H₂ gas entry. For this medium cycling, third and fourth fluid connections 63, 64 are arranged between the second bioreactor 3 and the vessel 4. The third fluid connection 63 fluidly connects a second outlet 37 of the second bioreactor 3 with a first inlet 44 of the vessel, the first inlet 44 being arranged in an upper part 41 of the vessel 4 in the height of the head space 42 of the vessel 4. A first pump 10 is arranged in the third fluid connection 63 for pumping second medium 33 from the second bioreactor 3 to the vessel 4. The fourth fluid connection 64 fluidly connects a first outlet 45 of the vessel 4 with a second inlet 36 of the second bioreactor 3, and allows reflow of third medium 43 from the vessel 4 to the second bioreactor 3. The flow of the third medium 43 to the second bioreactor 3 can, for example, be controlled with a second valve 82. Part of the second medium 33 is withdrawn from section B and brought to a pressure >p3 with the first pump 10 in order to finely spray it through a nozzle 12 into the head space 42 of the vessel 4 of section C. This fine distribution in the head space 42 of the vessel 4 with H₂ atmosphere ensures further enrichment of H₂ in the liquid phase. The inflow of H₂-saturated medium into the second bioprocess in section B can be regulated by the circuit via the first pump 10 and the second valve 82 using vl1 and vl2 (see FIG. 2). There will also be second microorganisms, i.e. Knallgas bacteria, in this cycle that can benefit from the different dissolved gas concentrations.

In section B there is a pressure p2 that is preferably lower than the pressure p3, which allows the dissolved H₂ to be better released into the surrounding medium. Here the CO₂ and O₂ input is increased by stirring with a second stirrer 31 and optional gas cycling between sections A and B via valve 81, arranged in an ninth fluid connection 69 fluidly connecting a third outlet 39 of the second bioreactor 3 arranged at the height of the head space 32 of the second bioreactor 3 and a second inlet 26 of the first bioreactor 2 arranged at the height of the head space 22 of the first bioreactor 2.

The pressure p1 in section A is preferably lower than in B and C, but preferably slightly above atmospheric pressure. In sections B and C, overpressures can be reduced or controlled with valves 83 and 84, if required.

The biomass generated in section B is preferably continuously extracted via a second pump 11 and a separator 6, once a sufficient cell density of the second microorganisms has been reached. In this process, second medium 33 containing the second microorganisms is withdrawn from the second bioreactor 3, the cells are separated from the second medium 33 using a separator 6, for example a continuous centrifuge, e.g. a disk stack centrifuge, or a hollow fiber membrane module, hydrolyzed in a hydrolyzer 7 and fed into the first bioreactor 2. For this purpose, a second fluid connection 62, e.g. a pipe or tube, fluidly connects a first outlet 35 of the second bioreactor 3 with a first inlet 24 of the first bioreactor 2. The separator 6 is, in relation to the flow direction of second medium 33, arranged after the pump 11, and the hydrolyser 7 is arranged after the separator 6. In this embodiment, second medium 33 separated from the cells is returned to the bioprocess of section B via an eighth fluid connection 68 fluidly connecting the part of the separator 6 with the separated second medium 33 to a fourth inlet 30 of the second bioreactor 3. Optionally, second medium can be removed from the circuit via a fifth valve 85 for reprocessing, for example, and reintroduced in section C. The extracted biomass is hydrolyzed with the hydrolyzer 7 and fed to the bioprocess in section A as an additional C and N source. At least part of the biomass can also be withdrawn and used as a separate bioproduct, e.g. fish food. Depending on the mode of operation of the bioprocess in section A (batch or continuous, for example), the biomass work-up can be carried out continuously or at intervals. In this manner, an estimated 20-60% of the expensive glucose can be saved in a lipid fermentation step and the C balance can be significantly shifted in the direction of higher lipid yields.

Fresh medium 103 can be introduced into the process in section C, e.g. at a third inlet 48 of the vessel 4. First microorganisms and/or first medium 23 can be withdrawn from the section A, for example via a third outlet 29 of the first bioreactor 2.

FIG. 2 shows a simplified scheme of the apparatus shown in FIG. 1, and shows gas flows (dashed lines) and liquid flows (solid lines) into and between sections A, B or C (see description to FIG. 1).

1. A process for the biotechnological production of a bioproduct, the process comprising:
a) a first bioprocess comprising cultivation of first microorganisms in a first bioreactor in a first medium at a first pressure (p1), the first bioprocess resulting in the formation of the bioproduct and carbon dioxide (CO₂),
b) a second bioprocess comprising cultivation of second microorganisms in a second bioreactor in a second medium at a second pressure (p2), the second bioprocess being a process producing biomass and consuming molecular oxygen (O₂), molecular hydrogen H₂, and at least part of the (CO₂) produced in the first bioprocess of step a),
c) electrolyzing water into O₂ and H₂, feeding at least part of the O₂ produced by electrolysis into the second bioprocess and/or into the first bioprocess, and feeding at least part of the H₂ produced by electrolysis into a vessel containing a head space and a third medium at a third pressure (p3), the third medium being identical to the second medium in the second bioreactor, and
d) feeding at least part of the H₂-containing third medium from the vessel to the second bioprocess,
wherein at least part of the biomass produced in the second bioprocess of step b) is used as a C-source for the first bioprocess of step a),
wherein the CO₂ produced in the first bioprocess is fed into the second bioprocess,
wherein the first microorganisms are yeast cells, and wherein the second bioprocess involves chemolithoautotrophic consumption of O₂, H₂ and CO₂.
wherein the first, second and third medium are in each case an aqueous medium that is suitable for supporting growth of the first microorganisms and the second microorganisms.

2. The process according to claim 1, wherein p3≥p2≥p1.

3. The process according to claim 1, wherein the first bioprocess is an aerobic bioprocess.

4. (canceled)

5. The process according to claim 1, wherein the second bioprocess is performed at a temperature between 40-55° C.

6. The process according to claim 1, wherein part of the second medium of the second bioreactor containing the second microorganisms is withdrawn from the second bioreactor, the biomass contained in the second medium is at least partly separated from the second medium, hydrolyzed and fed into the first bioprocess, and the second medium separated from the biomass is recycled into the second bioprocess.

7. The process according to claim 1, wherein part of the second medium from the second bioreactor is fed into the vessel and sprayed into the head space of the vessel, and the same amount of third medium is fed from the vessel to the second bioreactor.

8. The process according to claim 1, wherein part of the O₂ produced by electrolysis is fed into the first bioprocess.

9. The process according to claim 1, wherein the first microorganisms are microorganisms of the genus Rhodosporidium.

10. The process according to claim 1, wherein p1 is greater than atmospheric pressure.

11. The process according to claim 1, wherein the process is a continuous process.

12. An apparatus for the biotechnological production of a bioproduct according to the process of one of the preceding claims, the apparatus comprising:
a) a first bioreactor being configured for carrying out a first bioprocess, wherein the first bioprocess comprises the cultivation of first microorganisms in a first medium at a first pressure p1, and wherein the first bioprocess results in the formation of the bioproduct and carbon dioxide CO₂,
b) a second bioreactor being configured for carrying out a second bioprocess, wherein the second bioprocess comprises the cultivation of second microorganisms in a second medium at a second pressure p2, and wherein the second bioprocess is a process producing biomass and consuming molecular oxygen O₂, molecular hydrogen H₂, and at least part of the CO₂ produced in the bioprocess of step a),
c) a vessel being configured to contain a head space and a third medium at a third pressure p3, and
d) an electrolyzer for electrolyzing water into O₂ and H₂, and wherein the apparatus further comprises
a first fluid connection fluidically connecting a first outlet of the first bioreactor to a first inlet of the second bioreactor, for the transfer of CO₂ produced in the first bioreactor to the second bioreactor,
a second fluid connection fluidically connecting a first outlet of the second bioreactor to a first inlet of the first bioreactor, for the transfer of biomass produced in the second bioreactor to the first bioreactor,
a third fluid connection fluidically connecting a second outlet of the second bioreactor to a first inlet of the vessel, for the transfer of second medium to the vessel,
a fourth fluid connection fluidically connecting a first outlet of the vessel to a second inlet of the second bioreactor, for the transfer of third medium to the second bioreactor,
a fifth fluid connection fluidically connecting a first outlet of the electrolyzer to a third inlet of the second bioreactor, for the transfer of oxygen O₂ to the second bioreactor,
a sixth fluid connection fluidically connecting a first outlet of the electrolyzer to a third inlet of the first bioreactor, for the transfer of oxygen O₂ to the first bioreactor, and
a seventh fluid connection fluidically connecting a second outlet of the electrolyzer to a second inlet of the vessel, for the transfer of hydrogen H2 to the vessel,
wherein the first, second and third medium are in each case an aqueous medium that is suitable for supporting growth of the first microorganisms and the second microorganisms.

13. The apparatus according to claim 12, wherein, the second fluid connection comprises, between the first outlet of the second bioreactor and the first inlet of the first bioreactor, a separator for separating the second microorganisms from the second medium, and a hydrolyzer for hydrolyzing the second microorganisms separated from the second medium by the separator.

14. The apparatus according to claim 12, wherein the third fluid connection fluidically connecting the second outlet of the second bioreactor to the first inlet of the vessel, extends into an upper part of the vessel comprising the head space, the extension comprising a nozzle for atomizing second medium fed into the vessel.

15. The apparatus according to claim 12, wherein the first fluid connection fluidically connecting the first outlet of the first bioreactor to the first inlet of the second bioreactor, comprises a CO² concentrator for concentrating the CO₂ before its transfer to the second bioreactor.

16. The apparatus according to claim 12, wherein the first fluid connection fluidically connecting the first outlet of the first bioreactor to the first inlet for the second bioreactor, comprises a CO₂ storage tank for intermediately storing CO₂.
